(19) United States
(12) Patent Application Publication
(10) Pub. No.: US 2004/0156846 A1
(43) Pub. Date: Aug. 12, 2004

(54) Therapy via targeted delivery of nanoscale particles using L6 antibodies

(75) Inventors: Wolfgang Daum, Groton, MA (US); Gerald DeNardo, El Macerco, CA (US); Diane Ellis-Busby, Lancaster, MA (US); Alan Foreman, Epping, NH (US); Douglas U. Gwost, Shoreview, MN (US); Erik Schroeder Handy, Arlington, MA (US); Robert Ivkov, Marblehead, MA (US)

Correspondence Address: ALTERA LAW GROUP, LLC, 6500 CITY WEST PARKWAY, SUITE 100, MINNEAPOLIS, MN, 55344-7704, US

(73) Assignee: Triton Biosystems, Inc., Chelmsford, MA

(21) Appl. No.: 10/360,578

(22) Filed: Feb. 6, 2003

(51) Int. Cl. A61N 1/30; A61K 39/395

(52) U.S. Cl. 424/144.1; 604/20

(57) ABSTRACT

Methods for treating cells, diseased tissue, pathogens, or other undesirable matter involve the administration of a bioprobe (energy susceptive materials that are attached to the target-specific ligand chimeric L6 antibody) to a patient's body, body part, tissue, or body fluid (such as blood, blood plasma, or blood serum). An energy source provides energy to the bioprobe so as to destroy, rupture, or inactivate the target. Various energy forms, such as AMF, microwave, acoustic, or a combination thereof, created via a variety of mechanisms, may be used. The disclosed methods may be useful in the treatment of a variety of indications, including but not limited to, cancer of any type, such as bone marrow, lung, vascular, neuro, colon, ovarian, breast and prostate cancer.

### TECHNICAL FIELD

The present invention relates generally to therapeutic methods, and specifically, to therapeutic methods that comprise the administration of an energy susceptive material, that is attached to the target-specific ligand chimeric L6 antibody, to a patient's body, body part, tissue, or body fluid, and the administration of energy from an energy source, so as to destroy or inactivate the target.

### BACKGROUND

The time between the onset of disease in a patient and the conclusion of a successful course of therapy is often unacceptably long. Many diseases remain asymptomatic and evade detection while progressing to advanced, and often terminal, stages. In addition, this period may be marked by significant psychological and physical trauma for the patient due to the unpleasant side effects of even correctly prescribed treatments. Even diseases that are detected early may be most effectively treated only by therapies that disrupt the normal functions of healthy tissue or have other unwanted side effects.

One such disease is cancer. Despite considerable research effort and some success, cancer is still the second leading cause of death in the United States, claiming more than 500,000 lives each year according to American Cancer Society estimates. Traditional treatments are invasive and/or are attended by harmful side effects (e.g., toxicity to healthy cells), often making for a traumatic course of therapy with only modest success. Early detection, a result of better diagnostic practices and technology, has improved the prognosis for many patients. However, the suffering that many patients must endure makes for a more stressful course of therapy and may complicate patient compliance with prescribed therapies. Further, some cancers defy currently available treatment options, despite improvements in disease detection. Of the many forms of cancer that still pose a medical challenge, prostate, breast, lung, and liver claim the vast majority of lives each year. Colorectal cancer, ovarian cancer, gastric cancer, leukemia, lymphoma, melanoma, and their metastases may also be life threatening.

Conventional treatments for breast cancer, for example, typically include surgery followed by radiation and/or chemotherapy. These techniques are not always effective, and even if effective, they suffer from certain deficiencies. Surgical procedures range from removal of only the tumor (lumpectomy) to complete removal of the breast. In early stage cancer, complete removal of the breast may provide an assurance against recurrence, but is disfiguring and requires the patient to make a very difficult choice. Lumpectomy is less disfiguring, but can be associated with a greater risk of cancer recurrence. Radiation therapy and chemotherapy are arduous and are not completely effective against recurrence.

Treatment of pathogen-based diseases is also not without complications. Patients presenting symptoms of systemic infection are often mistakenly treated with broad-spectrum antibiotics as a first step. This course of action is completely ineffective when the invading organism is viral. Even if a bacterium (e.g., E. coli) is the culprit, the antibiotic therapy eliminates not only the offending bacteria, but also benign intestinal flora in the gut that are necessary for proper digestion of food. Hence, patients treated in this manner often experience gastrointestinal distress until the benign bacteria can repopulate. In other instances, antibiotic-resistant bacteria may not respond to antibiotic treatment. Therapies for viral diseases often target only the invading viruses themselves. However, the cells that the viruses have invaded and “hijacked” for use in making additional copies of the virus remain viable. Hence, progression of the disease is delayed, rather than halted.

For these reasons, it is desirable to provide improved and alternative techniques for treating disease. Such techniques should be less invasive and traumatic to the patient than the present techniques, and should only be effective locally at targeted sites, such as diseased tissue, pathogens, or other undesirable matter in the body. Preferably, the techniques should be capable of being performed in a single or very few treatment sessions (minimizing patient non-compliance), with minimal toxicity to the patient. In addition, the undesirable matter should be targeted by the treatment without requiring significant operator skill and input.

Immunotherapy is a rapidly expanding type of therapy used for treating a variety of human diseases including cancer, for example. The FDA has approved a number of antibody-based cancer therapeutics. The ability to engineer antibodies, antibody fragments, and peptides with altered properties (e.g., antigen binding affinity, molecular architecture, specificity, valence, etc.) has enhanced their use in therapies. Cancer immunotherapeutics have made use of advances in the chimerization and humanization of murine antibodies to reduce immunogenic responses in humans. High affinity human antibodies have also been obtained from transgenic animals that contain many human immunoglobulin genes. In addition, phage display technology, ribosome display, and DNA shuffling have allowed for the discovery of antibody fragments and peptides with high affinity and low immunogenicity for use as targeting ligands. All of these advances have made it possible to design an immunotherapy that has a desired antigen binding affinity and specificity, and minimal immune response.

The field of cancer immunotherapy makes use of markers that are over-expressed by cancer cells (relative to normal cells) or expressed only by cancer cells. The identification of such markers is ongoing and the choice of a ligand/marker combination is critical to the success of any immunotherapy. Immunotherapeutics fall into at least three classes: (1) deployment of antibodies that, themselves, target growth receptors, disrupt cytokine pathways, or induce complement or antibody-dependent cytotoxicity; (2) direct arming of antibodies with a toxin, a radionuclide, or a cytokine; (3) indirect arming of antibodies by attaching them to immunoliposomes used to deliver a toxin or by attaching them to an immunological cell effector (bispecific antibodies). Although armed antibodies have shown potent tumor activity in clinical trials, they have also exhibited unacceptably high levels of toxicity to patients.

The disadvantage of therapies that rely on delivery of immunotoxins or radionuclides (i.e., direct and indirect arming) has been that, once administered to the patient, these agents are active at all times. These therapies often cause damage to non-tumor cells and present toxicity issues and delivery challenges. For example, cancer cells commonly shed surface-expressed antigens (targeted by immunotherapeutics) into the blood stream. Immune complexes can be formed between the immunotherapeutic and the shed antigen. As a result, many antibody-based therapies are diluted due to the interaction of the antibody with these shed antigens rather than interacting with the cancer cells, and thereby reducing the true delivered dose. Thus, a “therapy-on-demand” approach that minimizes adverse side effects and improves efficacy would be preferable.

With thermotherapy, temperatures in a range from about 40° C. to about 46° C. (hyperthermia) can cause irreversible damage to disease cells. However, healthy cells are capable of surviving exposure to temperatures up to around 46.5° C. Elevating the temperature of individual cells in diseased tissue to a lethal level (cellular thermotherapy) may provide a superior treatment option. Pathogens implicated in disease and other undesirable matter in the body can also be destroyed via exposure to locally high temperatures.

Hyperthermia may hold promise as a treatment for cancer and other diseases because it induces instantaneous necrosis (typically called “thermo-ablation”) and/or a heat-shock response in cells (classical hyperthermia), leading to cell death via a series of biochemical changes within the cell. State-of-the-art systems that employ microwave or radio frequency (RF) hyperthermia, such as annular phased array systems (APAS), attempt to tune energy for regional heating of deep-seated tumors. Such techniques are limited by the heterogeneities of tissue and to highly perfused tissue. This leads to the as-yet-unsolved problems of “hot spot” phenomena in untargeted tissue with concomitant underdosage in the desired areas. These factors make selective heating of specific regions with such systems very difficult.

Another strategy that utilizes RF hyperthermia requires surgical implantation of microwave or RF based antennae or self-regulating thermal seeds. In addition to its invasiveness, this approach provides few (if any) options for treatment of metastases because it requires knowledge of the precise location of the primary tumor. The seed implantation strategy is thus incapable of targeting undetected individual cancer cells or cell clusters not immediately adjacent to the primary tumor site. Clinical success of this strategy is hampered by problems with the targeted generation of heat at the desired tumor tissues.

### SUMMARY OF THE INVENTION

Hyperthermia for treatment of disease using energy sources exterior to the body has been recognized for several decades. However, a major problem has been the inability to selectively deliver a lethal dose of heat to the cells or pathogens of interest.

In view of the above, there is a need for a method for treating diseased tissue, pathogens, or other undesirable matter that incorporates selective delivery of energy to a target within a subject's body. It is also desirable to have treatment methods that are safe and effective, short in duration, and require minimal invasion.

It is, therefore, an object of the present invention to provide a treatment method that involves the administration of energy susceptive materials that are attached to the a target-specific ligand, to a subject's body, body part, tissue, or body fluid, and the administration of an energy source to destroy, rupture, or inactivate the target.

It is another object of the present invention to administer the energy to a selected cell or tissue, to a subject's entire body, or extracorporeally to the subject's body.

The present invention pertains to a treatment method that comprises the administration of a bioprobe (energy susceptive particles that are attached to the target-specific ligand chimeric L6 antibody) to a subject, and administration of an energy source, to the bioprobe, after a prescribed period of time for the bioprobe to locate and attach to the markered target, a glycoprotein antigen, particularly the L6 antigen, so as to destroy or inactivate the target. The energy may be administered directly into the subject's body, body part, tissue, or body fluid (such as blood, blood plasma, or blood serum), or extracorporeally to the subject's body.

The above summary of the present invention is not intended to describe each illustrated embodiment or every implementation of the present invention. The figures and the detailed description that follow particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates a bioprobe configuration, according to an embodiment of the present invention;

FIG. 2 schematically illustrates target specific bioprobes bound to a disease cell surface, according to an embodiment of the present invention;

FIG. 3 schematically illustrates a therapy system, according to an embodiment of the present invention; and

FIG. 4 schematically illustrates an alternating magnetic field (AMF) therapy system, according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF EXEMPLARY EMBODIMENTS

1. Definitions

The term “susceptor”, as used herein, refers to a particle (optionally comprising a coating) of a material that, when exposed to an energy source, either heats or physically moves. Similarly, the term “magnetic susceptor” refers to such particles wherein the energy source to which the particles respond is an alternating magnetic field (AMF).

The term “ligand”, as used herein, refers to a molecule or compound that attaches to a susceptor (or a coating on the susceptor) and targets and attaches to a biological marker.

The term “bioprobe”, as used herein, refers to a composition comprising a susceptor and at least one ligand. The ligand acts to guide the bioprobe to a target.

The term “marker”, as used herein, refers to an antigen or other substance to which the bioprobe ligand is specific.

The term “target”, as used herein, refers to the matter for which deactivation, rupture, disruption or destruction is desired, such as a diseased cell, a pathogen, or other undesirable matter. A marker may be attached to the target. Breast cancer cells are exemplary targets.

The term “bioprobe system”, as used herein, refers to a bioprobe specific to a target that is optionally identified via a marker.

The term “indication”, as used herein, refers to a medical condition, such as a disease. Breast cancer is an exemplary indication.

The term “RF” (an abbreviation for radio frequency), as used herein, refers to a radio frequency in the range of about 0.1 Hz to about 900 MHz.

The term “AMF” (an abbreviation for alternating magnetic field), as used herein, refers to a magnetic field that changes the direction of its field vector periodically, typically in a sinusoidal, triangular, rectangular or similar shape pattern. The AMF may also be added to a static magnetic field, such that only the AMF component of the resulting magnetic field vector changes direction. It will be appreciated that an alternating magnetic field is accompanied by an alternating electric field and is electromagnetic in nature.

The term “energy source”, as used herein, refers to a device that is capable of delivering energy to the bioprobe's susceptor.

The term “duty cycle”, as used herein, refers to the ratio of the time that the energy source is on to the total time that the energy source is on and off in one on-off cycle.

2. The Targeted Therapy System

The targeted therapy system of the present invention involves the utilization of a bioprobe system in conjunction with an energy source to treat an indication.

2.1 The Bioprobe System.

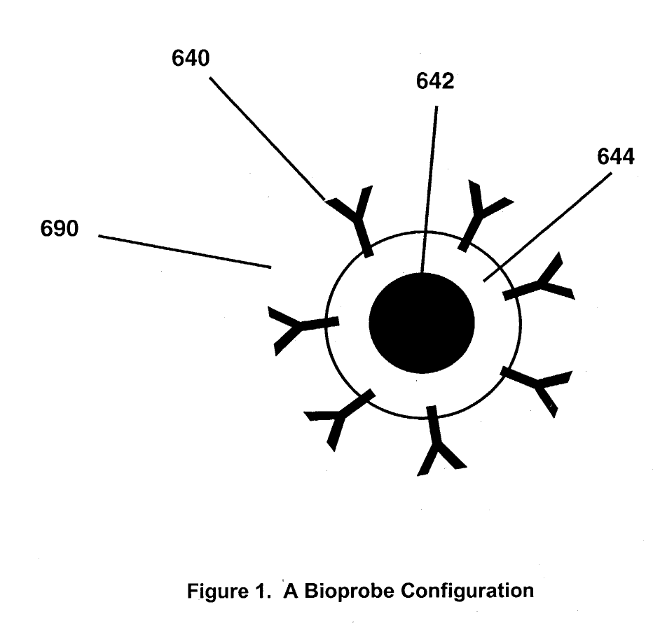

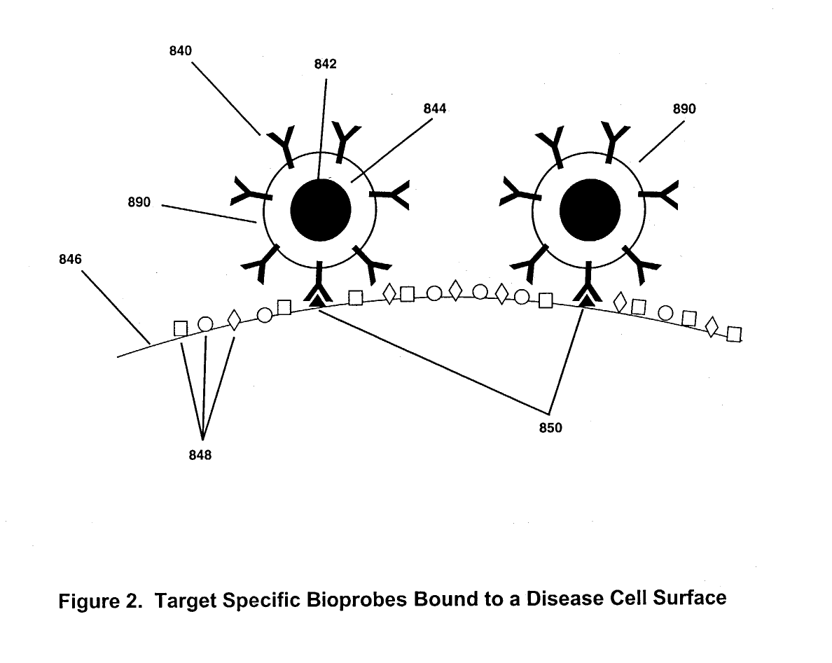

Various embodiments of the bioprobe system of the present invention are demonstrated via FIGS. 1 and 2. FIG. 1 illustrates a bioprobe configuration according to an embodiment of the present invention, wherein a bioprobe 690, comprises an energy susceptive particle, also referred to as a susceptor 642. The susceptor 642 may comprise a coating 644. At least one targeting ligand 640 may be located on an exterior portion of bioprobe 690. Targeting ligand 640 may be selected to seek out and attach to a target. Heat may be generated in the susceptor 642 when the susceptor 642 is exposed to an energy source. Coating 644 may enhance the heating properties of bioprobe 690, particularly if the coating 644 is a polymeric material.

FIG. 2 illustrates an embodiment of the present invention wherein a bioprobe 890, comprising a susceptor 842, which comprises a coating 844, is attached to a target (such as a cell) 846 by one or more targeting ligands 840. Cell 846 may express several types of markers 848 and 850. The specificity of bioprobe 890 is represented by its attachment to targeted marker 850 over the many other markers or molecules 848 on cell 846. One or more bioprobes 890 may attach to cell 846 via ligand 840. Ligand 840 may be adapted and bioprobe 890 may be designed such that bioprobe 890 remains externally on cell 846 or may be internalized into cell 846. Once bound to cell 846, the susceptor 842 is energized in response to the energy absorbed. For example, the susceptor 842 may heat up in response to the energy absorbed. The heat may pass through coating 844 or through interstitial regions to the cell 846, for example via convection, conduction, radiation, or any combination of these heat transfer mechanisms. The heated cell 846 becomes damaged, preferably in a manner that causes irreparable damage. When bioprobe 890 becomes internalized within cell 846, bioprobe 890 may heat cell 846 internally via convection, conduction, radiation, or any combination of these heat transfer mechanisms. When a sufficient amount of energy is transferred by bioprobe 890 to cell 846, cell 846 dies via necrosis, apoptosis or another mechanism.

According to one embodiment of the present invention, cancer cell-specific antibodies are linked to susceptors. The chimeric L6 antibody (ChL6) is preferable for use as the ligand in the methods of the present invention. The bioprobes containing this ligand target a glycoprotein antigen, particularly the L6 antigen. The L6 antigen is a 202 amino acid, cysteine-rich integral membrane glycoprotein that is highly expressed on lung, breast, colon, and ovarian carcinomas and minimally expressed on normal cells. The L6 antigen is a desirable target for therapeutic intervention due to its high level of expression on malignant cells. Furthermore, the L6 antigen is not shed. The L6 antigen is related to a number of cell surface proteins with similar predicted membrane topology that have been implicated in control of cell proliferation.

Chimeric L6 is an antibody chimera comprising a human IgG1 constant region and the variable region of the mouse antibody to L6. ChL6 antitumor antibody recognizes an epitope located in a 42-residue extracellular domain of a tumor-associated glycoprotein antigen of approximately 22 kDa. Both ChL6 and mouse L6 antibodies bind adenocarcinoma cells with the same avidity, but the ChL6 antibodies are 50 to 100 times more effective in mediating antibody dependent cellular toxicity in vitro.

Low tumor uptake of administered monoclonal antibodies has been a serious problem for many immunotherapies. ChL6 antibodies, on the other hand, target an abundant, non-shed antigen that is expressed on many human carcinomas. This results in high tumor uptake and localization in solid tumors in vivo, making ChL6 useful for treating a variety of cancers, for example radioimmunotherapy in breast cancer.

Chimeric L6 also induces vascular permeability leading to increased tumor uptake/penetration in vivo.

The methods of the present invention may be used to treat a variety of indications which include, but are not limited to, cancer of any type, such as bone marrow, lung, vascular, neuro, colon, ovarian, breast and prostate cancer.

2.2. The Energy Source

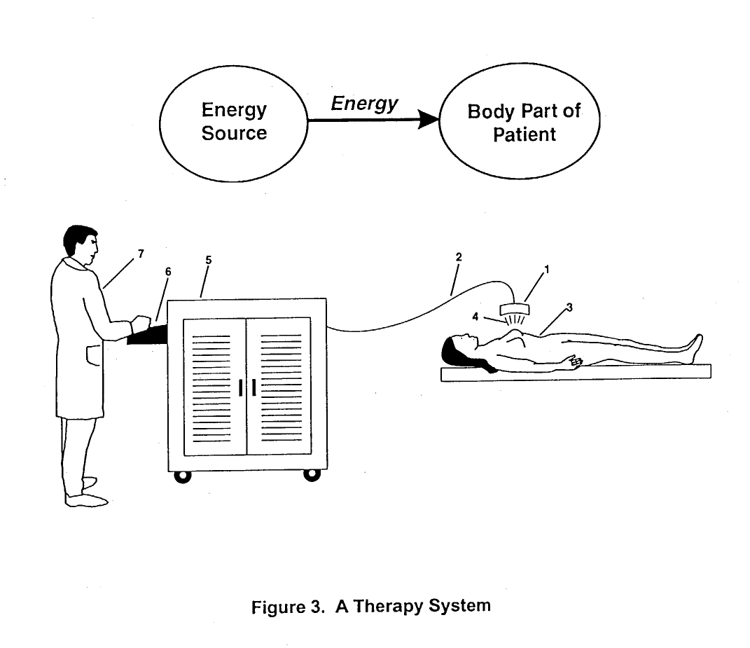

The energy source for use in the present invention includes any device that is able to provide energy to the susceptor that can convert that energy, for example to heat or mechanical motion. The bioprobe then transmits the heat or mechanical motion to the targeted cell and cells or tissue surrounding the targeted cell. FIG. 3 schematically illustrates an energy source that transmits energy to a subject's body or a body part. Some exemplary energy forms and energy sources useful herein are listed in Table I. The different forms of energy, for example AMF, microwave, acoustic, or a combination thereof, may be created using a variety of mechanisms, such as those listed in Table I. The table also lists those sections of the following description that are pertinent to the different energy forms and therapeutic mechanisms.

TABLE I
ENERGY SOURCES FOR ENERGIZING BIOPROBES

| CORRESPONDING | ENERGY | | THERAPEUTIC |
|---|---|---|---|
| SECTION BELOW | FORM | ENERGY SOURCE | MECHANISM |
| 2.2.1 (a) | AMF | Power Generator/Inductor | Induction Heating |
| 2.2.1 (b) | AMF | Power Generator/Inductor | Resonance Heating |
| 2.2.1 (c) | AMF | Power Generator/Inductor | Particle-Particle |
| | | | Friction Heating |
| 2.2.1 (d) | AMF | Power Generator/Inductor | Mechanical |
| | | | Displacement |
| 2.2.1 (e) | AMF | Power Generator/Inductor | Multi-Mechanism |
| 2.2.2 (a) | Microwave | Klystron, Cyclotron, Antennae, | Absorption Heating |
| | | Magnetron, Traveling Wave | |
| | | Tube, Backwards Oscillator, | |
| | | Cross Field Amplifier, | |
| | | Gyrotron, Injection Locked | |
| | | Magnetron | |
| 2.2.2 (b) | Microwave | Klystron, Cyclotron, Antennae, | Pulsed Heating |
| | | Magnetron, Travelling Wave | |
| | | Tube, Backwards Oscillator, | |
| | | Cross Field Amplifier, | |
| | | Gyrotron, Injection Locked | |
| | | Magnetron | |
| 2.2.2 (c) | Microwave | Klystron, Cyclotron, Antennae, | Resonance Heating |
| | | Magnetron, Traveling Wave | |
| | | Tube, Backwards Oscillator, | |
| | | Cross Field Amplifier, | |
| | | Gyrotron, Injection Locked | |
| | | Magnetron | |
| 2.2.2 (d) | Microwave | Klystron, Cyclotron, Antennae, | Multi-Mechanism |
| | | Magnetron, Traveling Wave | Heating |
| | | Tube, Backwards Oscillator, | |
| | | Cross Field Amplifier, | |
| | | Gyrotron, Injection Locked | |
| | | Magnetron | |
| 2.2.3 | Acoustic | Loudspeaker, Piezoelectric | Acoustic |
| | | Ultrasound Transducer | Absorption |
| 2.2.4 | AMF, | | Combination |
| | Microwave, | | Mechanism |
| | and | | |
| | Acoustic | | |
| 2.2.5 | AMF, | | Extracorporeal |
| | Microwave, | | |
| | and | | |
| | Acoustic | | |

In general, as illustrated in FIG. 3, operator 7 controls an energy generating device 5, for example via a console 6, which delivers energy, for example via a cable 2, to an energy source 1. Energy source 1 transmits energy 4 to the bioprobe's susceptor to heat or otherwise affect the targeted cell, and cells or tissue that surround the bioprobe in the subject.

It will be appreciated that the energy sources disclosed in patent applications having U.S. Ser. Nos. 10/176,950 and 10/200,082, the relevant portions of which are incorporated herein by reference, may also be used for heating the bioprobes of the present invention.

2.2.1 AMF

AMF energy may be used with a bioprobe to produce therapeutic mechanisms, such as heating, mechanical displacement, or various combinations thereof. Heating through the application of AMF to the bioprobe may be accomplished through a variety of mechanisms, such as induction, resonance, and particle-particle friction heating. These AMF energy forms are described hereinbelow.

2.2.1(a) AMF Induction Heating

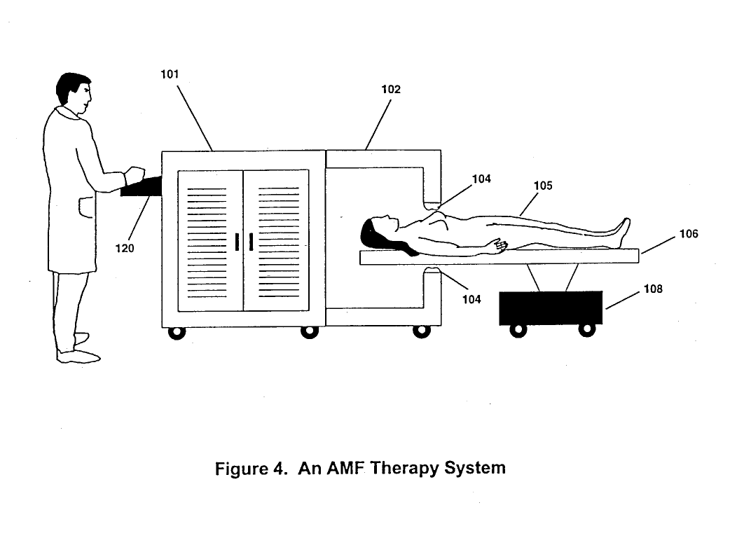

In one embodiment of the present invention, as illustrated in FIG. 4, the therapeutic system comprises an alternating magnetic field (AMF) generator, for example located within a cabinet 101, designed to produce an AMF that may be guided to a specific location within a subject 105 by a magnetic circuit 102. Subject 105 may lie upon an X-Y horizontal and vertical axis positioning bed 106. Positioning bed 106 can be positioned horizontally and vertically via a bed controller 108. The AMF generator produces an AMF in magnetic circuit 102 that exits magnetic circuit 102 at one pole face 104, passing through the air gap and the desired treatment area of subject 105, and reenters magnetic circuit 102 through the opposing pole face 104, thus completing the circuit. An operator or medical technician may control and monitor the AMF characteristics and bed positioning via a control panel 120. When the AMF is generated by an RF generator, the frequency of the AMF may be in the range of about 0.1 Hz to about 900 MHz.

Other approaches may be used to generate the AMF, and may provide a focused and/or a homogeneous field.

The magnetic susceptors for use herein typically are susceptible to AMF energy supplied by the energy source and heat when exposed to AMF energy; are biocompatible; and have surfaces that have (or can be modified to have) functional groups to which ligands can be chemically or physically attached. In one embodiment of the present invention, a susceptor having a magnetic core is surrounded by a biocompatible coating material. There are many possible combinations of core-coating materials. For example, gold as a coating material is particularly advantageous because it forms a protective coating to prevent a chemical change, such as oxidation, in the core material while being biocompatible. A gold coating can also be chemically modified to include groups for ligand linking. Further, gold serves as a good conductor for enhancing eddy current heating associated with AMF heating.

Types of magnetic susceptor cores that require a protective coating include iron, cobalt, and other magnetic metals. Iron and cobalt, for example, are susceptible to chemical changes, such as oxidation, and possess magnetic properties that are significantly changed due to oxidation. The use of a protective coating is especially preferred in embodiments where the core material may pose a toxic risk to humans and animals in vivo. Thus, the use of a gold coating material is particularly preferred to protect the core material from chemical attack, and to protect the subject from toxic effects of the core material.

In one particular embodiment of the present invention, the gold coating is chemically modified via thiol chemistry such that a chemical link is formed between the gold surface and a suitable ligand. For example, an organic thiol moiety can be attached to the gold, followed by linking the ligand to the organic thiol moiety using at least one silane, carboxyl, amine, or hydroxyl group, or a combination thereof. Other chemical methods for modifying the surface of the coating material may also be utilized.

In one embodiment of the present invention, nitrogen-doped Mn clusters are used as magnetic susceptors. These nitrogen-doped Mn clusters, such as MnN and MnₓN_{y}, where x and y are nonzero numbers, are ferromagnetic and comprise large magnetic moments. Calculations based on density-functional theory show that the stability and magnetic properties of small Mn clusters can be fundamentally altered by the presence of nitrogen. Not only are their binding energies substantially enhanced, but also the coupling between the magnetic moments at Mn sites remains ferromagnetic regardless of their size or shape.

In another embodiment, Nd₁₋ₓCaₓFeO₃ is used as a magnetic susceptor. The spontaneous magnetization of the weak ferromagnetism decreases with increasing Ca content or increasing particle size.

Other materials, such as superparamagnetic Co₃₆C₆₄, Bi₃Fe₅O₁₂, BaFe₁₂O₁₉, NiFe, CoNiFe, Co—Fe₃O₄, and FePt—Ag, may also be used as susceptors in the present invention.

2.2.1(b) AMF Resonance Heating

It is well known that atoms, molecules, and crystals possess resonance frequencies at which energy absorption is effectively achieved. In general, resonance heating offers significant advantages because the targeted material absorbs large quantities of energy from a relatively low power source. Thus, non-targeted materials, including body tissue, the resonant frequency of which differs from that of the targeted material, do not heat to the same extent. Accordingly, materials may be chosen to take advantage of a particular resonant frequency in the electromagnetic energy spectrum. A susceptor material may be selected such that the internal chemical bonds of the material may resonate at a particular frequency.

Resonance heating can also be achieved by exploiting interactions of AMF energy with materials that possess magnetic, electrical, or electric dipole structures on the atomic, molecular, or macroscopic length scales. In addition to the direct modes of heating described above, resonance heating may be used indirectly. In one embodiment of the present invention, materials for use as bioprobes are selected such that they possess magnetic or electric properties that will induce a shift in the resonance frequency of the tissue to which they become attached. Thus, the molecules of the tissue in close proximity to the bioprobes will heat preferentially in an applied energy field tuned to the appropriate frequency.

The energy can be applied to a targeted cell, targeted tissue, to the entire body, extracorporeally (outside of the subject's body) or in any combination thereof.

2.2.1(c) AMF Particle-Particle Friction Heating

Magnetic susceptors can also create physical or mechanical motion when they are exposed to AMF. This motion results in friction between the particles to create heat. In one embodiment of the present invention, particles having sizes in the range of about 10 nm to about 10,000 nm are exposed to an AMF frequency, e.g., at 60 Hz. More specifically, susceptors having sizes in the range of about 50 nm to about 200 nm are displaced 3 cm in distance and rotated up to 360° in one AMF cycle. The external magnetic forces required to mechanically displace the susceptors depend upon the anisotropy energy of the magnetic domains, size, and shape of the susceptors. At higher frequencies the particle displacement is reduced.

When a sufficiently high number of bioprobes are attached to the target, the susceptors make contact such that they generate heat through friction when mechanically displaced by the AMF. The displacement amplitude, and therefore heating efficiency, is larger at lower frequencies where induction heating is less efficient.

2.2.1(d) Mechanical Displacement

Energy for use in the methods of the present invention can also produce mechanical displacement of the bioprobes. At low bioprobe concentrations, the bioprobes do not touch each other, however, AMF induces bioprobes that are intimately attached to the targeted cells to vibrate, rotate, displace and otherwise create motion. This motion may disrupt the targeted cell or rupture the cell membrane of the targeted cells. One preferred frequency range for this effect is from about 1 Hz to about 500 Hz, although this effect may also be used with applied frequencies outside this range. At higher AMF frequencies, the displacement amplitude of the bioprobes is reduced and therefore the field strength can be increased to achieve the same effect. Examples of susceptors suitable for use in bioprobes for mechanical displacement include particles of Fe₂O₃ and Fe₃O₄, although other magnetic particles may also be used. The particle size may be in the range from about 5 nm to about 1 μm, although the particle size may also fall outside this range.

2.2.1(e) Multi-Mechanism

Any combination of the mechanisms discussed in Section 2.2.1 herein can also be utilized in the methods of the present invention. In addition, the subject's body may be utilized in the creation of additional therapeutic heating. Body tissue heats by eddy currents induced by the AMF. Eddy currents flow around the whole body, or around organs or organ parts, which are electrically conducting and possess a certain minimal magnetic susceptibility. An incremental therapeutic heating can be captured by taking advantage of this effect. Thus, a dual mechanism that includes AMF heating of the susceptors and eddy current heating of body tissue may also be useful herein.

2.2.2 Microwave Heating

The microwave heating for use herein may be accomplished through a variety of heating mechanisms, such as microwave absorption, pulsed microwave, resonance microwave, or a combination thereof, all at frequencies of 900 MHz and above. These mechanisms are described hereinbelow.

2.2.2(a) Microwave Absorption Heating

Certain particles, which are typically metallic but can also be non-metallic, can be heated at frequencies in the upper megahertz and gigahertz region of the electromagnetic wave spectrum by simple energy absorption. In an embodiment of the present invention involving extracorporeal heating, microwaves can be focused directly into the blood/blood serum/blood plasma flowing through the energy source to heat the bioprobe.

2.2.2(b) Pulsed Microwave Heating

Because microwaves are directly absorbed by tissue, as with AMF heating, the duty cycle significantly affects the heating of a subject's body or body part. Therefore, it is preferable to pulse the microwave energy because the conduction of heat from particles to tissue differs from tissue to tissue heating. This is particularly applicable in embodiments in which an organ is heated extracorporeally, and the tissue is cooled by the flow of blood through the tissue. For example, when microwave susceptible bioprobes are attached to liver cancer cells and the liver is laid open to expose it to microwave energy, the blood and blood vessels will also heat, but such heat is efficiently removed. The ‘on’ time of the radiation would typically be in the range of about 0.1 second to about 1200 seconds and the ‘off’ time would be in the range of about 0.1 second to about 1200 seconds. It will be appreciated that pulsed microwave heating may also apply to resonance microwave heating and microwave absorption heating.

2.2.2(c) Resonance Microwave Heating

Resonance microwave heating is utilized in the same manner as the AMF resonance heating described hereinabove.

2.2.2(d) Multi-Mechanism Microwave Heating

Microwave absorption, pulsed microwave, and resonance microwave heating mechanisms may be utilized in any combination in the therapeutic methods of the present invention.

2.2.3 Acoustic Absorption

The therapeutic mechanism of the present invention may also use absorption of acoustic energy. Acoustic waves, for example in the range of about 500 kHz to about 16 MHz, propagate through tissue. In one embodiment of the present invention, nanotubes fabricated from MoS₂, W₁₈O₄₉, NiCl₂, NbS₂, GaSe or single crystal C₆₀ are used as susceptors. These susceptors typically have an inner diameter of about 1 nm to about 10 nm, outer diameter of about 2 nm to about 20 nm, and a length of up to about 20 nm. When the frequency of an acoustic wave is in resonance with mechanical virbrational resonance of these nanotubes, the nanotubes vibrate and they either heat or explode so as to disrupt, rupture or inactivate the target.

2.2.4 Combination Mechanism

Any combination of the AMF, microwave, and acoustic energy providing mechanisms, described hereinabove, may be used to provide the necessary energy for the therapeutic methods of the present invention.

2.2.5 Extracorporeal Therapy

In one embodiment of the present invention, a subject is treated via extracorporeal therapy. The bioprobes may be used to lyse, denature, or otherwise damage the disease material by removing material from the subject, exposing the material to an energy source, and returning the material to the body. The bioprobes may be introduced into the subject's body or body part and then removed from the subject along with the material that is being extracted. The bioprobes may be separated from the material that is extracted after the treatment. Alternatively, the bioprobes are introduced to the extracted material while the extracted material is outside of the subject's body or body part. For example, where the extracted material is the subject's blood, the bioprobes may be introduced to the vascular circulating system or into the blood circulating outside of the body, prior to exposure to an energy source.

In embodiments where the bioprobe/target complexes that are carried primarily in the blood serum or blood plasma are targeted, the blood serum or blood plasma may be separated extracorporeally from the other blood components, exposed to an energy source so as to destroy or inactivate the target, and recombined with the other blood components prior to returning the blood to the subject's body. The bioprobes may be introduced into the vascular circulating system, the blood circulating outside of the body, or the blood serum or blood plasma after it is separated.

In another embodiment, the bioprobes may be contained in a vessel or column through which the blood circulating outside of the body or the blood serum or blood plasma flows. The vessel or column may be exposed to an energy source so as to destroy or inactivate the targeted cells or antigens prior to returning the blood to the subject's body.

The advantages of providing energy to the bioprobes extracorporeally include the ability to heat to higher temperatures and/or heat more rapidly to enhance efficacy while minimizing heating and damage to surrounding body tissue, and the ability to reduce exposure of the body to the energy from the energy source. In embodiments where the bioprobes are introduced into the blood circulating outside of a subject's body, the blood serum, or blood plasma that is extracted from the body, bioprobes need not be directly introduced into the body, and higher concentrations of bioprobes can be introduced to the target. Further, the portion of the subject that is being treated extracorporeally can be cooled externally, using a number of applicable methods, while energy is provided to the bioprobes without mitigating the therapeutic effect. In addition, the cooling may take place before, and/or after the administration of energy.

The treated bioprobes and the associated targets need not be returned to the subject's body. For example, if the bioprobes and the associated targets are contained in blood extracted from a subject, the treated bioprobes and the associated targets may be separated from the blood prior to returning the blood to the subject's body. In embodiments where the bioprobes contain a magnetic component, the bodily fluids containing the bioprobes and associated targets are passed through a magnetic field gradient in order to separate the bioprobes and the associated targets from the extracted bodily materials. In doing so, the amount of susceptors and treated disease material returned to the subject's body is reduced.

In another embodiment of extracorporeal treatment, the tissue selected for heating is completely or partially removed from a subject's body for example, during an open surgical procedure. The tissue can remain connected to the body or can be dissected and reattached after the therapy. In yet another embodiment, the tissue can be removed from the body or body part of one donor subject and transplanted to that of a recipient subject after the therapy.

While the above description of the invention has been presented in terms of a human subject, it is appreciated that the invention may also be applicable to treating other subjects, such as mammals, cadavers and the like.

As noted above, the present invention is applicable to methods for treating diseased tissue, pathogens, or other undesirable matter that involve the administration of energy susceptive materials, that are attached to the target-specific ligand ChL6, to a subject's body, body part, tissue, or body fluid, and the administration of an energy source to the energy susceptive materials. The present invention should not be considered limited to the particular embodiments described above, but rather should be understood to cover all aspects of the invention as fairly set out in the attached claims. Various modifications, equivalent processes, as well as numerous structures to which the present invention may be applicable will be readily apparent to those skilled in the art to which the present invention is directed upon review of the present specification. The claims are intended to cover such modifications and devices.

We claim:

1. A therapeutic method, comprising:
a) administering at least one bioprobe to at least a portion of a subject comprising a target; and
b) administering energy from an energy source to the at least one bioprobe combined with the target; and
wherein the bioprobe comprises a susceptor and a chimeric L6 antibody.

2. A therapeutic method according to claim 1, wherein the target is associated with a cancer.

3. A therapeutic method according to claim 2, wherein the target comprises a marker and wherein the marker is a glycoprotein antigen.

4. A therapeutic method according to claim 3, wherein the marker glycoprotein is an L6 antigen.

5. A therapeutic method according to claim 1, wherein the chimeric antibody to marker L6 antigen comprises a human IgG1 constant region and a variable region of the mouse antibody to L6 antigen.

6. A therapeutic method according to claim 1, wherein the energy is administered to provide heating, and wherein the energy is in the form of AMF, microwave, acoustic, or any combination of thereof.

7. A therapeutic method according to claim 6, wherein the energy form is microwave having a frequency of at least 900 MHz, AMF having a frequency of from about 0.1 Hz to 900 MHz, acoustic having a frequency of from about 500 kHz to about 16 MHz, or any combination thereof.

8. A therapeutic method according to claim 6, wherein the energy is pulsed.

9. A therapeutic method according to claim 8, wherein the energy ‘on’ pulse times are in the range from about 0.1 seconds to about 1200 seconds, and the ‘off’ pulse times are in the range from about 0.1 seconds to about 1200 seconds.

10. A therapeutic method according to claim 1, wherein the energy source provides energy in a frequency range in which the susceptor possesses a resonance frequency, causing the energy absorption of the susceptor to be enhanced at said resonance frequency.

11. A therapeutic method according to claim 10, wherein the energy source is pulsed.

12. A therapeutic method according to claim 1, wherein the portion of the subject is extracted from the subject's body prior to extracorporeal administration of energy.

13. A therapeutic method according to claim 12, wherein the extracted portion of the subject is returned to the subject's body or is transplanted to a recipient's body after the administration of energy.

14. A therapeutic method according to claim 12, wherein the extracted portion of the subject is cooled before, during or after the administration of energy.

15. A therapeutic method according to claim 14, wherein the susceptor is magnetic, and wherein the magnetic susceptor is removed from the extracted portion via a magnetic force after the administration of energy.

16. A therapeutic method according to claim 1, further comprising surgically opening the subject, and wherein the portion of the subject is tissue laid open to provide access for bringing the energy source close to the targeted tissue.

17. A therapeutic method according to claim 7, wherein the susceptor comprises a group of nitrogen-doped Mn clusters, MnN, MnₓN, Mn-doped GaN, Nd₁₋ₓCaₓFeO₃, superparamagnetic Co₃₆C₆₄, Bi₃Fe₅O₁₂, BaFe₁₂O₁₉, NiFe, CoNiFe, Co—Fe₃O₄, FePt—Ag, or a combination thereof, and wherein the susceptor is heated via AMF.

18. A therapeutic method according to claim 7, wherein the susceptor comprises a magnetic core having a gold coating, and wherein the energy is AMF heating.

19. A therapeutic method according to claim 19, wherein the susceptor comprises an organic thiol moiety that is attached to the gold coating, and wherein the bioprobe ligand is attached to the organic thiol moiety using at least one silane, carboxyl, amine, hydroxyl group or a combination thereof.

20. A therapeutic method according to claim 7, wherein the energy is in the form of AMF and heats the bioprobe, and wherein the AMF further induces eddy current heating of the portion of the subject.

21. A therapeutic method according to claim 1, wherein the energy is administered to cause mechanical motion of the susceptor, and wherein the energy is in the form of acoustic energy.

22. A therapeutic method according to claim 21, wherein the susceptor is a nanotube fabricated from MoS₂, single crystal C₆₀, W₁₈O₄₉, NiCl₂, NbS₂, or GaSe, or a combination thereof.

23. A therapeutic method according to claim 21, wherein the acoustic energy has frequencies in the range from about 500 kHz to about 16 MHz.
